(19) United States
(12) Patent Application Publication
(10) Pub. No.: US 2026/0134598 A1
(43) Pub. Date: May 14, 2026

(54) METHOD AND SYSTEM FOR EFFICIENT AND EFFECTIVE ADAPTATION, CUSTOMIZATION OF DIFFUSION FOUNDATION MODELS IN MEDICAL IMAGING

(71) Applicant: Siemens Healthineers AG, Forshheim (DE)

(72) Inventors: Mariappan S. Nadar, Plainsboro, NJ (US); Bingyu Xin, Piscataway, NJ (US); Radu Miron, Iasi (RO); Mahmoud Mostapha, Princeton, NJ (US); Nirmal Janardhanan, Alpharetta, GA (US); Rainer Schneider, Höchstadt (DE); David Grodzki, Erlangen (DE); Omar Darwish, London (GB); Tobias Würfl, Erlangen (DE); Till Hülnhagen, Erlangen (DE); Jens Gühring, Erlangen (DE); Boris Mailhe, Plainsboro, NJ (US)

(21) Appl. No.: 19/191,324

(22) Filed: Apr. 28, 2025

Related U.S. Application Data
(60) Provisional application No. 63/717,906, filed on Nov. 8, 2024.

(30) Foreign Application Priority Data
Nov. 8, 2024 (EP) 24465591.6

(51) Int. Cl. G06T 11/00; G06V 10/84; G16H 30/40

(52) U.S. Cl. CPC G06T 12/20; G06V 10/84; G16H 30/40; G06T 2211/424; G06T 2211/441

(57) ABSTRACT

Systems and methods for image reconstruction of magnetic resonance data using a conditioned plug-and-play (PnP) method, where a diffusion foundation model is used as implicit image prior for efficient and effective adaptation/customization for specialized clinical use-cases. By attaching an Adapter network to the diffusion foundation model, the method adds extra specialized constraints into PnP iterations.

### CROSS REFERENCE TO RELATED APPLICATIONS

This application claims priority to U.S. provisional application Ser. No. 63/717,906, filed Nov. 8, 2024, and European Patent Application 24465591.6, filed Nov. 8, 2024, both of which are entirely incorporated by reference.

### FIELD

This disclosure relates to medical imaging.

### BACKGROUND

Magnetic resonance imaging, or MRI, is a noninvasive medical imaging test that can generate detailed images of almost every internal structure in the human body, including, for example organs, bones, muscles, and blood vessels. Traditional MRI is slow due to the need for sequential data acquisition, often resulting in long scan times that can cause patient discomfort and motion artifacts. Acceleration techniques aim to overcome these limitations using methods like parallel imaging, compressed sensing, and deep learning-based reconstructions. Parallel imaging, such as SENSE and GRAPPA, utilizes multiple receiver coils to acquire data simultaneously, reducing scan time. Compressed sensing leverages sparsity in MRI images to reconstruct high-quality images from undersampled data, significantly shortening acquisition time. The shortened acquisition time, however, provides less information for image reconstruction.

Deep learning-based approaches have been proposed that use neural networks to enhance image reconstruction, improving speed and accuracy. In alternative fields, generative artificial intelligence (AI) tools such as large language models for text and diffusion models for audio, images and video have seen tremendous progress in recent years. Applying these generative AI tools to MR reconstruction and/or refinement may be more efficient, improve the speed, and/or improve the accuracy.

### SUMMARY

By way of introduction, the preferred embodiments described below include methods, systems, instructions, and/or computer readable media for efficient and effective adaptation customization of diffusion foundation models in medical imaging.

In a first aspect, a method for image reconstruction of medical imaging data, the method comprising: acquiring a medical image of a patient; iteratively refining the medical image using a conditioned diffusion PnP model comprising a diffusion PnP model and an adapter, wherein the diffusion PnP model includes measurement during reverse diffusion steps that is carried out after a correction step that accounts for an inaccurate estimation resulting from computing a proximal solution, wherein the adapter comprises a neural network that adds conditions to the diffusion PnP model via finetuning; and outputting a refined medical image.

In a second aspect, a system for image reconstruction of medical imaging data, the system comprising: a medical imaging system configured to acquire medical imaging data; a conditioned diffusion PnP model comprising a diffusion PnP model and an adapter, wherein the diffusion PnP model includes measurement during reverse diffusion steps that is carried out after a correction step that accounts for an inaccurate estimation resulting from computing a proximal solution, wherein the adapter comprises a neural network that adds conditions to the diffusion PnP model via finetuning; wherein the conditioned diffusion PnP model is further configured to input the medical imaging data and output a reconstructed medical image.

In a third aspect, a method for training a conditioned diffusion PnP model, the method comprising: providing a pre-trained diffusion PnP model and an adapter network configured to input a control map; training the conditioned diffusion PnP model comprising the pre-trained diffusion PnP model and the adapter network, wherein weights of the pretrained diffusion PnP model are locked during the training which keeps the pretrained diffusion PnP model's weights unchanged while the adapter network's weights are changed; and outputting a trained conditioned diffusion PnP model.

Any one or more of the aspects described above may be used alone or in combination. These and other aspects, features and advantages will become apparent from the following detailed description of preferred embodiments, which is to be read in connection with the accompanying drawings. The present invention is defined by the following claims, and nothing in this section should be taken as a limitation on those claims. Further aspects and advantages of the invention are discussed below in conjunction with the preferred embodiments and may be later claimed independently or in combination.

### BRIEF DESCRIPTION OF THE DRAWINGS

The components and the figures are not necessarily to scale, emphasis instead being placed upon illustrating the principles of the embodiments. Moreover, in the figures, like reference numerals designate corresponding parts throughout the different views.

FIG. 1 depicts an example magnetic resonance apparatus according to an embodiment.

FIG. 2 depicts an example generative diffusion process for image processing according to an embodiment.

FIG. 3 depicts an example method for reconstructing/refining a medical image using a conditioned diffusion PnP model according to an embodiment.

FIG. 4 depicts an example of a conditioned diffusion workflow according to an embodiment.

FIG. 5 depicts an example algorithm for Diffusion PnP with an Adapter according to an embodiment.

FIG. 6 depicts an example architecture for a conditioned diffusion PnP model according to an embodiment.

FIG. 7 depicts example images generated by the conditioned diffusion PnP model according to an embodiment.

FIG. 8 depicts an example evaluation unit configured to implement the conditioned diffusion PnP model according to an embodiment.

FIG. 9 depicts an example U-Net architecture according to an embodiment.

FIG. 10 depicts an example artificial neural network according to an embodiment.

FIG. 11 depicts an example convolutional neural network according to an embodiment.

FIG. 12 depicts an example method for training a conditioned diffusion PnP model according to an embodiment.

### DETAILED DESCRIPTION

Embodiments described herein provide systems and methods that provide a conditioned plug-and-play (PnP) method (Conditioned Diffusion PnP model) for medical imaging reconstruction/refinement, where a diffusion foundation model is used as an implicit image prior for efficient and effective adaptation/customization for specialized clinical use-cases. An adapter network is attached to the diffusion foundation model that adds extra specialized constraints into the PnP iterations.

For medical image reconstruction, supervised learning methods have been extensively used. Recently, self-supervised approaches have been proposed that eliminate the need for ground truth. Despite their success, these methods lack generalizability and data acquired with diverse acquisition protocols may pose issues to generating accurate and efficient reconstruction. In an example, generative models (in a PnP framework) have recently emerged as a robust method for medical image reconstruction. PnP approaches to solving inverse problems in complex MRI data apply diffusion-based generative priors. In such a scheme, a diffusion model is used to model the prior distribution and may be used in a number of inverse tasks such as denoising or super-resolution without the need to train individual models for each task. This has led to exceptional performance of such diffusion based inverse solvers in CT or complex MRI data while retaining perceptual quality and reconstruction faithfulness.

These diffusion models have shown outstanding capabilities in modeling image distributions, making them expressive image priors for solving inverse problems. However, existing methods require a high number of Neural Function Evaluations (NFEs) for high-quality results, making them impractical in certain applications. Recent methods, referred to as diffusion plug-and-play (Diffusion-PnP) models provide a better trade-off between perceptual quality and NFEs. For Diffusion-PnP, a diffusion foundation model captures rich image priors from underlying data distributions. From a Bayesian perspective, the diffusion foundation models learn the a priori probability density function of the images. The diffusion foundation model solves the Bayesian inverse problem, tantamount to drawing posterior samples (and/or computing the posterior mean) from the posterior density function that is a product of the likelihood function (physical and statistical model of the imaging system) and the learnt a priori probability density function. However, while these models provide excellent reconstruction/refinement, the customization and adaptation of these generalized solutions for special/custom clinical use-cases have not been explored.

Embodiments provide for customization and adaptation of Diffusion-PnP by adding a control adapter network that adds spatial conditioning controls to the foundation diffusion model. Compared to conditional diffusion models that need to have all data paired for training, or finetuning which could lead to catastrophic forgetting, the Conditioned Diffusion PnP model only needs to finetune its adapter for a specific subset of the dataset. Further, the adapter may be enabled or disabled, depending on the task. The combination of the Diffusion-PnP and the adapter improves the prior robustness and speeds up Diffusion-PnP convergence for reconstruction.

Compared to discriminative denoisers, existing diffusion models provide better image perceptual quality. Despite this advantage, slow inference speed hinders the usability of such methods in clinical applications. The Conditioned Diffusion PnP model with DDIM sampling as provided by certain embodiments leads to less than 100 NFEs providing lower inference times and decreased resource requirements. Further, the Conditioned Diffusion PnP model leads to strong generalization capabilities and high-quality image reconstruction. It can also be used for super-resolution (or other restoration tasks) if adjustment to the data consistency is provided. In a particular example using diffusion weighted imaging, a higher SNR low b value image provides guidance in the PnP algorithm. Embodiments may also accommodate different user preferences for the reconstructed image impression through tuning Diffusion PnP hyperparameters, for example X and (, which control the strength of the condition guidance and the level of noise injected at each timestep. Embodiments further allow for multiple adapters to be trained and used, either independently or in combination depending on the characteristics of the input medical imaging data. This is possible due to the addition involved in the skip connections of the diffusion prior's decoder. For example, besides low b value images for DWI, noise maps, guidance/auxiliary images, derived maps from guidance/auxiliary images (e.g. edge maps, segmentation masks etc.), different acquisition protocol images (different contrasts, diffusion directions, structural scans such as T2w images in DWI), adjacent slices etc. may be used. Embodiments are also flexible, for example where the same prior may be used with or without the adapter at inference, depending on the user. In an example, when using a high b value DWI adapter if restoration for images that are not high b value DWI is desired, the use of the adapter may be switched off.

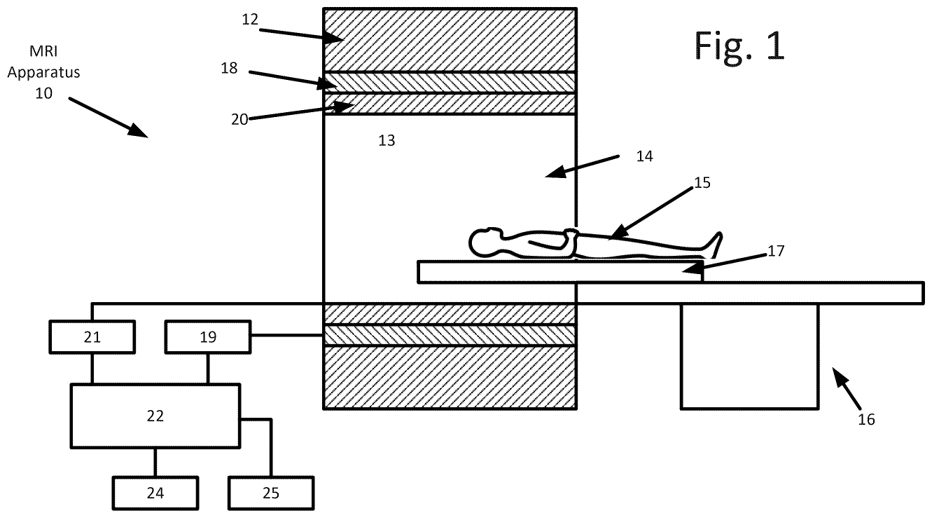

The examples provided below are in a MRI context but may be used for different modalities and protocols where applicable. FIG. 1 depicts an example magnetic resonance apparatus 10. The magnetic resonance apparatus 10 includes a magnetic unit 11 that includes a main magnet 12 for the generation of a main magnetic field 13. In addition, the magnetic resonance apparatus 10 includes a patient receiving area 14 for receiving a patient 15. The patient receiving area 14 may be cylindrical in design and cylindrically surrounded by the magnetic unit 11 in a circumferential direction. Different designs of the patient receiving area 14 may be used. The patient 15 may be pushed into the patient receiving area 14 by a patient positioning device of the magnetic resonance apparatus 10. The patient positioning device includes a patient table 17 for this purpose that is configured to be movable within the patient receiving area 14.

The magnetic unit 11 also includes a gradient coil unit 18 for the generation of gradient pulses that are used for location coding during imaging. The gradient coil unit 18 is controlled by a gradient control unit 19 of the magnetic resonance apparatus 10. The magnetic unit 11 also includes a radio frequency antenna unit 20, that may be configured as a body coil permanently integrated into the magnetic resonance apparatus 10. The radio frequency antenna unit 20 is controlled by a radio frequency antenna control unit 21 of the magnetic resonance apparatus 10 and emits RF transmission pulses into an examination area during a magnetic resonance measurement, that is essentially formed by a patient receiving area 14 of the magnetic resonance apparatus 10. As a result, atomic nuclei which are present in the main magnet field generated by the main magnet 12 are excited. Magnetic resonance signals are generated by relaxation of the excited atomic nuclei. The radio frequency antenna unit 20 is configured to receive magnetic resonance signals.

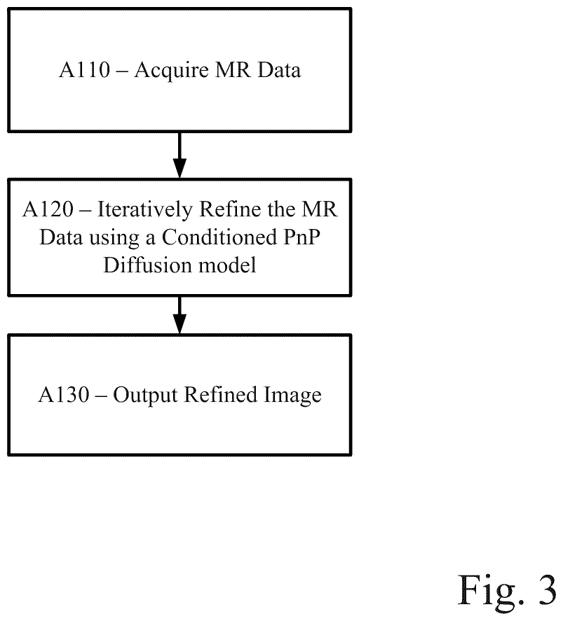

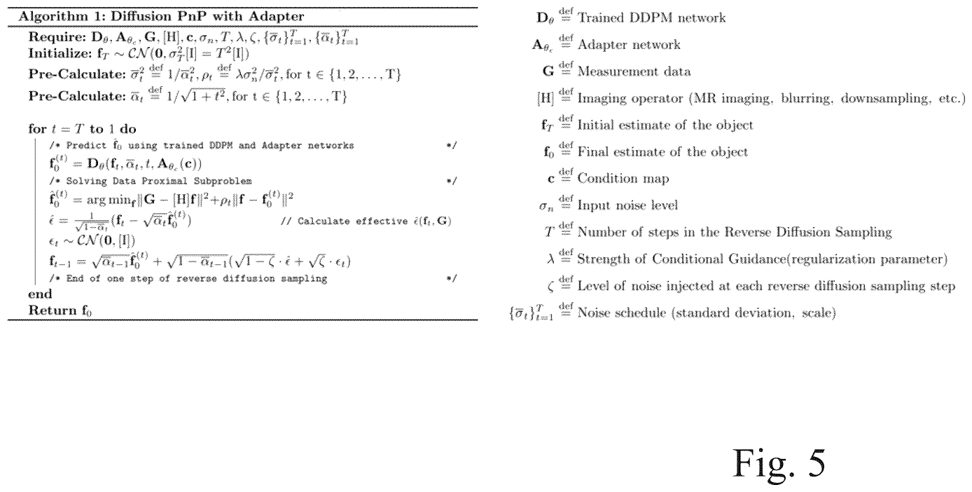

The magnetic resonance apparatus 10 includes a system control unit 22 for controlling the main magnet 12, the gradient control unit 19 and for controlling the radio frequency antenna control unit 21. The system control unit 22 controls the magnetic resonance apparatus 10, such as, for example, performing a predetermined imaging magnetic resonance measurement. The system control unit 22 may be configured to execute a computer-implemented method for generating magnetic resonance images, as shown in FIGS. 3, 5, and/or 12.

In addition, the system control unit 22 includes an evaluation unit 60 not shown here in more detail for evaluating the magnetic resonance signals that are recorded during the magnetic resonance examination. The evaluation 60 includes a processor 62, memory 64, and interface 66. Furthermore, the magnetic resonance apparatus 10 may also include a user interface for a medical operator that is connected to the system control unit 22. Control information such as, for example imaging parameters, as well as reconstructed magnetic resonance images may be displayed on a display unit 24, for example on at least one monitor. Furthermore, the user interface has an input unit 25 by which information and/or parameters may be entered by the medical operator during a measurement process.

The MR imaging device 36 is configured by the imaging protocol to scan a region of a patient 15. For example, in MR, such protocols for scanning a patient 11 for a given examination or appointment include diffusion-weighted imaging (acquisition of multiple b-values, averages, and/or diffusion directions), and other imaging procedures which depict the anatomy with different contrasts.

Diffusion-weighted imaging (DWI) is one of the key elements in multi-parametric magnetic resonance imaging (mpMRI) used in oncological studies as well as in many disease processes, such as cerebral ischemia, brain tumors, focal liver lesions, and Parkinson's disease. DWI allows detection and characterization of alterations of diffusion processes within the body tissue. Acquisition of a limited number of images with different b values and the subsequent fitting of the signal intensity decay observed at increasing b values is the easiest approach used to analyze DWI data. For example, in prostate cancer (PCa), high b values increase cancer conspicuity and reduce the influence of capillary perfusion. Despite this advantage, high b value images may also suffer from susceptibility artifacts as well as a decreased signal-to noise ratio (SNR). To address these issues, approaches such as multi-shot EPI and the reduced field-of-view (rFOV) technique have been proposed. Despite increasing the image quality, they lead to prolong acquisition times. One other approach is to under sample the k-space data. With emerging deep learning techniques and compressed sensing (CS), even higher acceleration factors may be obtained without compromising image quality. Alternative MR protocols may be used to acquire the MR data. Such MR protocols include a variety of pulse sequences tailored to specific diagnostic purposes. Common examples include T1-weighted imaging (for anatomical detail and tissue contrast), T2-weighted imaging (highlighting fluid or edema), fluid-attenuated inversion recovery (FLAIR, sensitive to pathology), diffusion tensor imaging (DTI, assessing white matter integrity), functional MRI (fMRI, detecting brain activation patterns), and susceptibility-weighted imaging (SWI, visualizing blood and iron content). Additional protocols include MR angiography (MRA, imaging blood vessels), spectroscopy (MRS, analyzing tissue metabolites), and gradient-echo sequences (GRE, rapid imaging, sensitive to susceptibility effects). Each of these protocols may have different sets of parameters and settings providing hundreds or thousands of possibilities of combinations when performing a patient depending on the equipment, modality, operator, purpose, etc. An adapter, as described below, may be provided for each of these clinical uses or applications where data is available.

The control unit 22 is configured to reconstruct an image using the acquired MRI data from the imaging procedure. Image reconstruction may be performed by the system 10 or other computing devices. MRI image reconstruction is the process of converting raw data from an MRI scan into a clinical image. It is a critical step in the MRI process, as the quality of the reconstructed image can affect the accuracy of the diagnosis. Noisy images or those lacking detail, for example, may be difficult to interpret and may result in inaccurate diagnoses.

Embodiments provide a conditioned plug-and-play (PnP) method, where a diffusion model is used as implicit image prior for accurate reconstruction of complex MRI data, for example high b-value complex DWI MRI data. Attaching an Adapter network to the diffusion model, extra constraints are added into PnP iterations providing further guidance when reconstructing the image thereby providing a more efficient and accurate output. The model is referred herein to as a Conditioned Diffusion PnP model.

In embodiments described herein, image reconstruction/restoration uses a generative deep learning framework, for example a diffusion model included in the Conditioned Diffusion PnP model, for reconstructing/restoring images from acquired MRI data. The generative deep learning model utilizes prior knowledge either with (supervised) or without (unsupervised) knowledge of a specific reconstruction task. By decoupling learning of the prior knowledge from the reconstruction task, the diffusion model may overcome existing issues of costly training and poor robustness to varied scan parameters. Inverse imaging problems-such as image reconstruction, super-resolution, and image deblurring require algorithms to estimate clear, detailed images from incomplete, noisy, or otherwise degraded data. While traditional diffusion models effectively capture sophisticated image priors, their reliance on training with entire images makes them computationally intensive and memory-demanding. Additionally, they require substantial training datasets, which can be challenging or impractical to obtain, particularly in medical imaging contexts or other specialized domains.

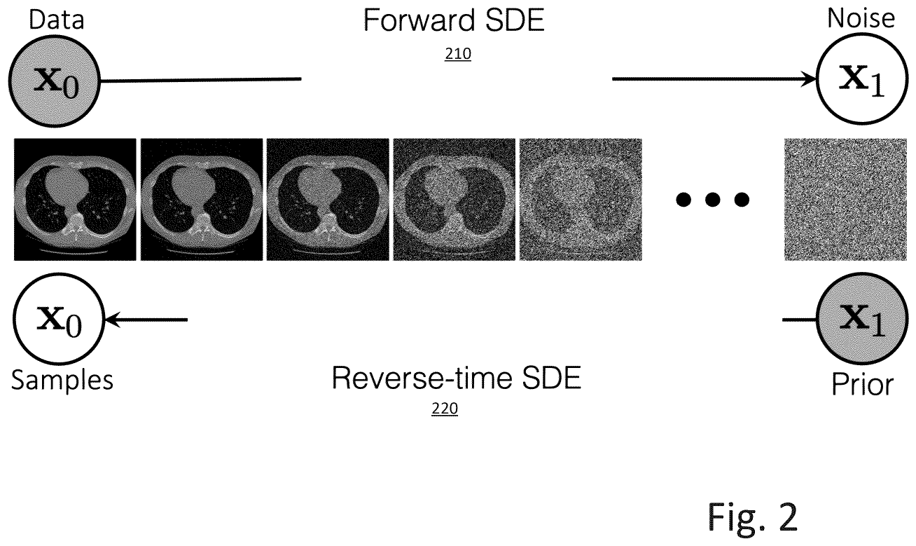

FIG. 2 depicts an example of an example generative diffusion process for image processing including a forward process 210 and a reverse process 220 (also referred to as the inference stage). The goal of the diffusion model is to learn the diffusion process for a given dataset, such that the process can generate new elements that are distributed similarly as the original dataset. In the forward stochastic differential equation (SDE) noise is added to the input image over and over again until the image is practically all noise. At each step, the diffusion model learns how to map images to their corresponding noise-free measurements. In the reverse step, the learned diffusion model is used to recover the data by reversing this noising process. Image reconstruction in MRI is a similar inverse problem that attempts to find an image from noisy scan measurements. To solve the inverse problem a forward model is defined that maps noisy MR images to their corresponding noise-free measurements. As measurements become noisier (for example as scan time is reduced) or less complete (for example when using increased acceleration), the resulting image reconstruction problem becomes highly ill-posed, meaning it has no stable, unique solution. In such situations the acquired measurements are said to be sparse, i.e., they are generally insufficient to uniquely specify a finite-dimensional approximation of the sought-after object, even in the absence of measurement noise or errors related to modeling the imaging system. False structures may arise due to the reconstruction method incorrectly estimating parts of the object that either did not contribute to the observed measurement data or cannot be recovered in a stable manner, a phenomenon that is referred to as hallucinations. Hallucinations may be resolved by incorporating information about the distribution of probable images, so-called prior knowledge. The reconstructed image balances maximizing both the likelihood that explains measurements, and the prior, that is, the probability that is a valid medical image. In embodiments described herein, the diffusion models capture rich image priors from underlying data distributions. From a Bayesian perspective, the diffusion models learn the a priori probability density function of the images. Solving the Bayesian inverse problem is tantamount to drawing posterior samples (and/or computing the posterior mean) from the posterior density function that is a product of the likelihood function (physical and statistical model of the imaging system) and the learnt a priori probability density function.

Embodiments provide a conditioned plug-and-play (PnP) method, where a diffusion model is used as implicit image prior for accurate reconstruction of MRI data. An adapter 440 is integrated into the diffusion model that adds extra constraints into PnP iterations. Compared to conditional diffusion models that need to have all data paired for training, or finetuning which could lead to catastrophic forgetting, The Adapted Diffusion PnP only needs to finetune its adapter 440 for a specific subset of the dataset. The adapter 440 in the Adapted Diffusion PnP may also be enabled or disabled, depending on the task.

FIG. 3 depicts an example method for reconstructing/refining a medical image using a conditioned diffusion PnP model. The method is performed by the system 10 of FIG. 1 or another system. The method is performed in the order shown or other orders. Additional, different, or fewer acts may be provided. In an example, a selection step may be used to indicate which adapters are to be used when reconstructing the image.

At act A110, a medical image of a patient 15 is acquired. The medical image may be acquired using an MRI scanning system 10 such as described in FIG. 1. Alternatively, the medical image may be provided from another source such as a database or previous scan. The medical imaging data may be acquired using an accelerated sequence. In an embodiment, an MRI system 10 acquires k-space measurements that are used to generate an initial reconstructed image that is input into the reconstruction/refinement process as described below. Different protocols may be used to acquire the MR data. For MRI, different protocols/sequences include T1-weighted imaging (for anatomical detail and tissue contrast), T2-weighted imaging (highlighting fluid or edema), fluid-attenuated inversion recovery (FLAIR, sensitive to pathology), diffusion tensor imaging (DTI, assessing white matter integrity), functional MRI (fMRI, detecting brain activation patterns), and susceptibility-weighted imaging (SWI, visualizing blood and iron content). Additional protocols include MR angiography (MRA, imaging blood vessels), spectroscopy (MRS, analyzing tissue metabolites), and gradient-echo sequences (GRE, rapid imaging, sensitive to susceptibility effects). The type of protocol/sequence/parameters may influence which adapter is used in the conditioned diffusion PnP model during reconstruction. Different adapters may be trained and applied for different uses. For example, the scan may be performed using GRAPPA (Generalized Auto calibrating Partially Parallel Acquisition) which accelerates image acquisition by using multiple receiver coils and under-sampling k-space and its corresponding noise map. The adapter may be configured with this information. Multiple adapters may be used, where the outputs may be weighted. For example a first adapter and a second adapter may be used. The two adapters output may be combined where the first adapter 440 is weighted at a certain level, for example 25%. The second adapter 440 would therefore make up the remaining 75% of the input to the diffusion foundation model. In a particular embodiment, a low B value may be used that is generally very high SNR image. Compared to the high B value, the high B value has lower SNR in general, in most of the diffusion weighted acquisitions. The system may reconstruct the low B using the foundation model and the plug and play reconstruction. But then when you are doing the reconstruction for a high B value, the low B high SNR low B may be input and used to improve the high B value. In another embodiment, a specific object or region is scanned of the patient. A mask of the object/region is used as the basis for the adapter, for example a segmentation of the mask from previous adjustment scans.

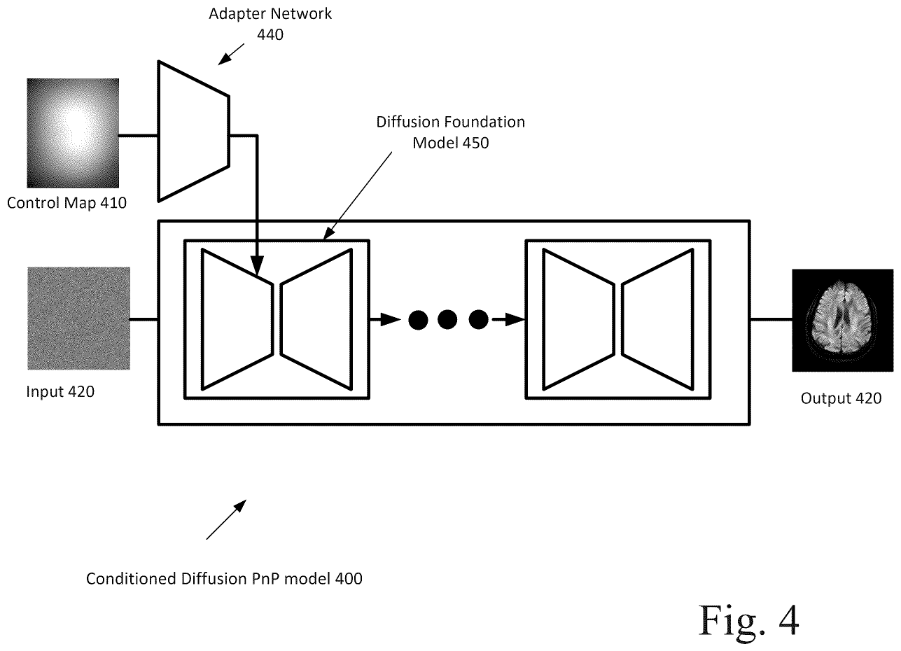

At act A120, the medical image is iteratively refined using the conditioned diffusion PnP Model including the foundation diffusion PnP model and the adapter network. The adapter network adds specialized constraints into the PnP iterations. FIG. 4 depicts an example of the diffusion workflow. In FIG. 4, the Adapter Network 440 and the Diffusion Foundation Model 450 make up the Conditioned Diffusion PnP model 400. The Diffusion Foundation Model 450 is at least in part provided by a pretrained diffusion PnP model. The Adapter Network 440 inputs a control map 410 that is used to condition (provide guidance to) the diffusion foundation model 450 which converts the input 420 to the output 420 refined image.

In an embodiment, the pretrained foundation diffusion model 450 (Diffusion PnP Model) is trained and applied to remove noise to predict a next state of the iterative process and measurement data (G) is incorporated by solving a data proximal subproblem, the measurement data (G) applied to the next state to ensure consistency. The diffusion PnP model 450 includes measurement during reverse diffusion steps, which is based on Denoising Diffusion Implicit Model DDIM and supports fast sampling. This measurement is carried out after a correction step that accounts for the inaccurate estimation resulting from computing the proximal solution. As a result of this process, the medical images are restored/refined to improve the quality of the images by mitigating noise, artifacts, or missing data. In a reverse process 210 of the diffusion PnP model, an image is generated using the learned probability density function of contrast weighted MR image data while being constrained by a data consistency term G that represents expected/known measurements.

Recovering the clean image f from its noisy measurement g=Hf+n, is equivalent to the following optimization problem:

$$\hat{f} = arg {min}_{f} \frac{1}{2} {‖ g - Hg ‖}^{2} + λ ϕ ( f )$$

where H is the degradation matrix and n is the additive Gaussian noise of standard deviation σₙ. PnP methods use variable splitting algorithms such as ADMM or half-quadratic splitting (HQS) and incorporate Gaussian denoisers. By using, for example, the HQS algorithm, the following subproblems are derived to be solved iteratively:

$${z}_{k} = {arg min}_{z} \frac{1}{2 {( \sqrt{\frac{λ}{μ}} )}^{2}} {‖ z - {f}_{k} ‖}^{2} + ϕ ( z )$$

$${f}_{k - 1} = {arg min}_{f} {‖ g - Hf ‖}^{2} + μ {σ}_{n}^{2} {‖ f - {z}_{k} ‖}^{2}$$

The subproblem with the prior term is a Gaussian denoising problem that is solved by the pretrained diffusion model together with adequate control maps, while the data term is tackled with a closed-form solution or a first-order proximal operator:

$${\hat{f}}_{0}^{( t )} \approx {f}_{0}^{( t )} - {\frac{{\overline{σ}}_{t}^{2}}{2 λ σ}}_{n}^{2} {∇}_{{f}_{0}^{( t )}} {‖ G - [ H ] {f}_{0}^{( t )} ‖}^{2}$$

that depends on H. The measurement data (G) may include measurements/linear transform of known features of the region or objects being scanned. For example, the measurement data (G) may include a ratio of the sizes or distances of or between two different features. This measurement is carried out after a correction step that accounts for the inaccurate estimation resulting from computing the proximal solution. The measurement data (G) is incorporated by solving the data proximal subproblem.

In an embodiment, the model uses DDPM. In an embodiment, in the reverse process, sampling is adapted from Deep Diffusion Implicit Models (DDIM). DDIM accelerates the sampling process of diffusion models by using non-Markovian diffusion processes. This approach allows for faster generation of high-quality images while maintaining the same training objective as traditional diffusion models. Implicit models focus on representing functions implicitly rather than explicitly. Instead of defining a mathematical formula directly, the implicit model defines a set of equations that describe the relationship between inputs and outputs without specifying the exact function.

The sampling process in DDIM involves sampling from the prior distribution and then iteratively sampling from the conditional distributions. This process is faster than traditional diffusion models because it does not require simulating the entire Markov chain. The number of NFEs, e.g. the total number of times the neural network needs to be called during the sampling process to generate a new image, is typically significantly lower in a DDIM compared to a standard DDPMs due to DDIM's more efficient non-Markovian diffusion process, resulting in faster generation times with fewer computations required. For example, fewer than 50 or 100 NFEs may be required to provide an acceptable output.

In an embodiment, a quadratic sampling technique is used. Sampling involves iteratively refining an image from a noisy initialization by stepping backward through a predefined sequence of time steps. The choice of these steps significantly impacts the efficiency and quality of image reconstruction. In a quadratic sampling scheme, the time steps are spaced according to a quadratic function, meaning the interval between successive steps increases quadratically as the sampling process progresses. This contrasts with uniform or geometric schedules, where the time steps are either equally spaced or decrease exponentially. The quadratic approach provides finer resolution in the early stages of denoising, when large noise components must be accurately removed, while allowing larger steps in later stages when the image structure has already stabilized. The use of this approach ensures that the early steps focus more on fine-grained denoising while later steps consolidate the reconstructed image. Different sampling techniques within diffusion models, like DPM-Solver or optimized ODE solvers, may also be used to adjust the required NFE. In an embodiment, the method may accommodate different user preferences for the reconstructed image impression through tuning Diffusion PnP hyperparameters, for example X and (, that control the strength of the condition guidance and the level of noise injected at each timestep.

An example of the algorithm is provided in FIG. 5. In Figure the denoised image is first predicted using the prior and the adapter. The low b value image is used as condition map. E[f₀|fₜ] is estimated via Tweedie's formula. If the adapter 440 is not to be used, the algorithm may ignore feature-maps coming from the adapter 440. Next the data proximal subproblem is solved to incorporate the measurement. To complete the sampling process, noise is added back in according to the next noise level indicated by the noise scheduler.

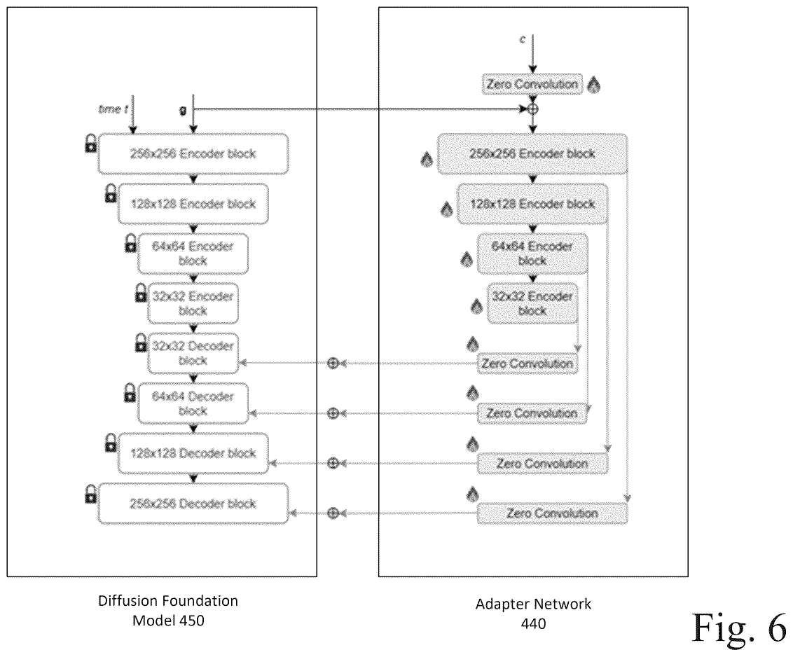

The adapter 440 is a neural network that adds spatially localized input conditions to the pretrained diffusion model via finetuning. The architecture of the adapter 440 is similar if not nearly identical to the encoder of the foundation diffusion model. An example of the adapter 440 architecture is provided in FIG. 6 which depicts the diffusion foundation model 450, the adapter network 440, and the connections therebetween. In FIG. 6, the control map c is first passed through a Zero-Conv layer. Skip connections to the frozen decoder of the pretrained diffusion prior are backed-up by other Zero-Conv layers. Thus, at the start of the training, strong noise is prevented from altering the feature-maps of deeper layers. In an example, the control map c is the low-b value DWI. Alternative control maps c may include information such as noise maps, guidance/auxiliary images, derived maps from guidance/auxiliary images (e.g. edge maps, segmentation masks etc.), different acquisition protocol images (different contrasts, diffusion directions, structural scans such as T2w images in DWI), adjacent slices, etc.

Referring back to the algorithm of FIG. 5, if D_{θ}=(Enc, Dec; θ) is the pretrained prior with frozen parameters θ, then for the Adapter 440, Aa, =(Enc; θ_{c}), with trainable parameters θ_{c}. Enc, Dec are the encoder and decoder structures in the U-Net of the foundation diffusion model 450. Several instances of zero-initialized layers, Z( ) are used to connect features from D_{θ} with features from A_{θ}_{_{c}}. Zero initialization is used to halt the influence of harmful noise into the hidden states of the trainable neural network when the training starts. If g is an image and c is a control map, the complete adapter 440 computes:

$$f := ControlNet ( g , c ; θ , {θ}_{c} , {θ}_{z 1} , {θ}_{z 2} ) = {D}_{θ} ( g ; θ ) + Z ( {A}_{{θ}_{c}} ( g + Z ( c ; {θ}_{z 1} ) ; {θ}_{c} ) ; {θ}_{z 2} )$$

As depicted in FIG. 6, the Adapter 440 duplicates the architecture of the pretrained diffusion model 450 to create a separate parallel path. The original model is “locked” (frozen weights) while the duplication is trained so that it can learn new conditional behaviors. The locked network ensures that the original generative capability remains unchanged. Meanwhile, the trainable copy is modified to accept an extra conditional input (e.g., a Canny edge map) and learn how to influence the generation process accordingly. Zero convolution layers, e.g., convolutional layers initialized with zero weights and zero bias are placed at the start of each block in the trainable adapter. Because these layers initially produce zero output, the layers do not affect the image generation at the beginning of training. Over time, as the model learns, the weights adapt and begin to incorporate the conditional information. In an embodiment, a zero convolution layer is simply a standard convolutional layer (e.g., 1×1 or 3×3) whose weights and biases are initialized to zero. This means that at initialization, the layer outputs all zeros regardless of the input, and it effectively does nothing when training begins. The zero convolution layers are critical for training because the goal is to add new conditional inputs (e.g., edge maps, depth maps) to the already trained diffusion model without changing its behavior initially. If the new layers had random weights, the layers could disrupt the generation process from the start and destabilize training. Instead, by starting from zero weights, the model behaves exactly like the original at the beginning, the new conditional input has no influence at first, the influence of the condition is gradually learned as training progresses, and this allows model to retain the benefits of pretraining while slowly learning how to incorporate new control signals. This design prevents disruptive interference with the pretrained model early in training and makes the training process stable. It essentially allows the model to learn conditional influence additively on top of the pretrained behavior. There is a branch that processes the condition (e.g., an edge map) and injects this information into each block of the U-Net of the foundation diffusion model. Each block receives features from both the locked and trainable copies and fuses them. This way, conditional guidance influences every layer of the image generation process-from low-level texture to high-level structure.

Different adapters 440 may be trained on various types of inputs. For example, an input control map may include or relate to low b value images for DWI, noise maps, guidance/auxiliary images, derived maps from guidance/auxiliary images (e.g. edge maps, segmentation masks etc.), different acquisition protocol images (different contrasts, diffusion directions, structural scans such as T2w images in DWI), adjacent slices, among others. Adapters 440 may be provided for specific equipment, patients, hospitals, operators, such as variations in scanner hardware, imaging sequences, and scanning parameters. Adapters 440 may be provided for any combination of scan settings or parameters. Additional information such as sensory information like respiratory curves or detected motion information may also be used by an adapter. In an embodiment, non-image related information such as patient related elements may be used. For instance patient demographic parameters, for example weight/fat content etc., might have a certain impact on image impression as the fat content in the body changes. Textual information or diagnostic values may be used in an adapter 440 by first transforming the textual information into an image map.

In addition, multiple adapters 440 may be applied by concatenating or otherwise combining the outputs. After training, the Conditioned diffusion PnP Model 400 may be applied to new images in order to generate images that precisely follow a structure or condition provided by the adapter 440.

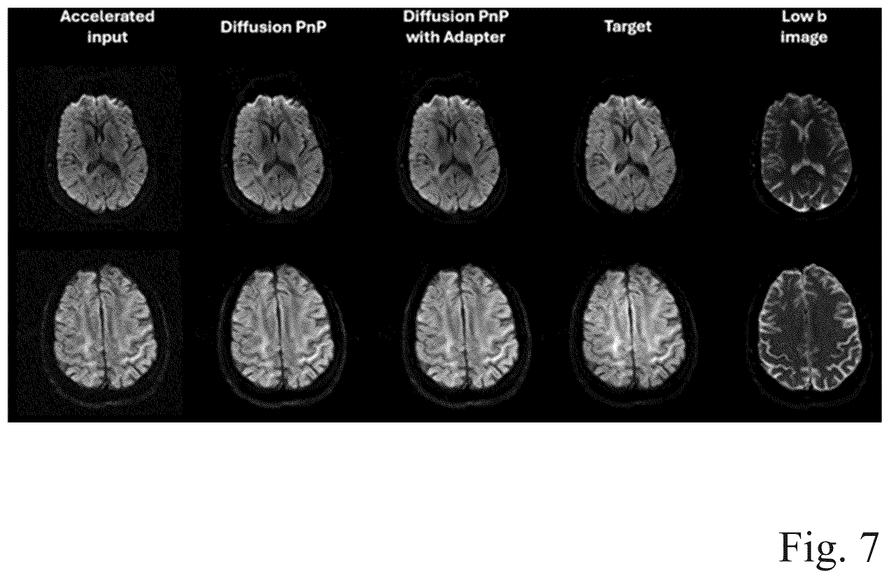

The iterative reconstruction/refinement process includes a plurality of steps for iteratively denoising/refining the image. After a number of iterations, the Conditioned diffusion PnP Model 400 estimates a refined image that at act A130 is output for display, analysis, or further processing. FIG. 7 depicts examples of a comparison of diffusion prior with and without adapter 440 on accelerated scans. For the images of FIG. 7, accelerated scans are simulated by dropping repetitions. An accelerated scan of factor 12 is given as input. 40 NFEs were used for both methods. Finer structures are recovered when using the adapter. In an embodiment, a 3D volume may be reconstructed by using 2D slices. The reconstruction processing may proceed slice by slice and use a previous reconstructed slice reconstruction to guide the next slice.

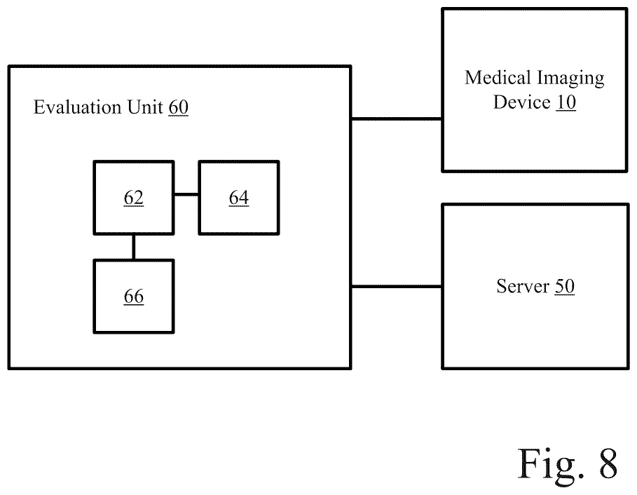

FIG. 8 depicts a system for training and/or implementing the Conditioned diffusion PnP Model 400. The system is configured to perform the method of FIG. 3 and other acts described herein. In FIG. 8, an evaluation unit 60 includes a processor 62, memory 64, and interface 66. In an embodiment, the evaluation unit 60 is configured for reconstructing/refining a medical image using an adapted Diffusion PnP model as described herein. The evaluation unit 60 is in communication with a medical imaging device 10, for example as described in FIG. 1, and a server 50. The server 50 may perform similar tasks as the evaluation unit 60 and/or may provide additional processing, storage, or analysis for example using a cloud based platform.

In an embodiment, the medical imaging device 10 is an MR imaging device 10, for example, as described above in FIG. 1. The MR system 10 of FIG. 1 includes an MR scanner or system, a computer based on data obtained by MR scanning, a server, or another processor. The MR imaging device 10 is only exemplary, and a variety of MR scanning systems may be used to collect the MR data. The MR imaging device 10 (also referred to as a MR scanner or image scanner) is configured to scan a patient 15. The MR imaging device 10 scans a patient 15 to provide k-space measurements (measurements in the frequency domain) using a specific protocol. The protocol and k-space measurements may be stored in the memory 64 and implemented by the processor 62.

The processor 62 may include an image processor 62 that generates images using a machine learning network (machine learning model). The image processor 62 is a general processor, digital signal processor, three-dimensional data processor, graphics processing unit, application specific integrated circuit, field programmable gate array, artificial intelligence processor, digital circuit, analog circuit, combinations thereof, or another now known or later developed device for image generation. The image processor 62 is a single device, a plurality of devices, or a network. For more than one device, parallel or sequential division of processing may be used. Different devices making up the image processor may perform different functions. In one embodiment, the image processor 62 is also a control processor or other processor of the imaging device. Other image processors of the imaging device or external to the imaging device may be used. The image processor 62 is configured by software, firmware, and/or hardware to process the data acquired by the imaging device and output one or more images.

The instructions for implementing the processes, methods, and/or techniques discussed herein are provided on non-transitory computer-readable storage media or memories, such as a cache, buffer, RAM, removable media, hard drive, or other computer readable storage media, for example the memory 64. The instructions are executable by the processor or another processor. Computer readable storage media include various types of volatile and nonvolatile storage media. The functions, acts or tasks illustrated in the figures or described herein are executed in response to one or more sets of instructions stored in or on computer readable storage media. The functions, acts or tasks are independent of the instructions set, storage media, processor or processing strategy and may be performed by software, hardware, integrated circuits, firmware, micro code, and the like, operating alone or in combination. In one embodiment, the instructions are stored on a removable media device for reading by local or remote systems. In other embodiments, the instructions are stored in a remote location for transfer through a computer network. In yet other embodiments, the instructions are stored within a given computer, CPU, GPU, or system. Because some of the constituent system components and method steps depicted in the accompanying figures may be implemented in software, the actual connections between the system components (or the process steps) may differ depending upon the manner in which the present embodiments are programmed.

In an embodiment, the processor 62 implements one or more machine learning networks that are stored in the memory 64. In general, a trained machine learning network mimics cognitive functions that humans associate with other human minds. In particular, by training based on training data the machine learning network is able to adapt to new circumstances and to detect and extrapolate patterns. Another term for “trained machine learning network” is “trained function”. In general, parameters of a machine learning network can be adapted by means of training. In particular, supervised training, semi-supervised training, unsupervised training, reinforcement learning and/or active learning can be used. Furthermore, representation learning (an alternative term is “feature learning”) can be used. In particular, the parameters of the machine learning networks can be adapted iteratively by several steps of training. In particular, within the training a certain cost function can be minimized. In particular, within the training of a neural network the backpropagation algorithm can be used. In particular, a machine learning network may comprise a neural network, a support vector machine, a decision tree and/or a Bayesian network, and/or the machine learning network can be based on k-means clustering, Q-learning, genetic algorithms, and/or association rules. In particular, a neural network can be a deep neural network, a convolutional neural network, or a convolutional deep neural network. Furthermore, a neural network can be an adversarial network, a deep adversarial network, and/or a generative adversarial network. The machine learning networks may be based upon or use existing methods while being improved upon by the innovative architecture and training mechanisms, in particular the use of the adapter 440 to condition the output of the model.

In an embodiment, the processor 22 implements a diffusion process for training and configuring the model. The diffusion process includes forward diffusion and reverse diffusion. Forward diffusion is used to add noise to the input image using a schedule which determines how much noise is added at the given step t. Reverse diffusion consists of multiple steps in which a small amount of noise is removed at every step. In an embodiment, the diffusion models use a modified U-Net architecture. At a high level, the modified U-Net includes an encoder, a bottleneck, and a decoder, forming a symmetric “U” shape. The encoder (downsampling path) captures hierarchical features by applying a sequence of convolutional blocks and spatial downsampling. Each encoder block extracts increasingly abstract features while reducing spatial resolution. The bottleneck sits at the center of the U and may include residual and attention blocks. These blocks allow the model to perform global reasoning across the latent representation. In particular, the attention mechanism helps correlate distant parts of the image. The decoder (upsampling path) reconstructs spatial resolution by using transposed convolutions or interpolation followed by convolutional layers. It mirrors the encoder's structure and includes skip connections from each encoder block. These skip connections pass high-resolution features directly to corresponding decoder layers, preserving local details and improving image fidelity. Each block in the U-Net may also incorporate time-step embeddings—representing the current diffusion step—and in certain embodiments text condition embeddings from a transformer-based text encoder (e.g., CLIP or OpenCLIP). These embeddings may be fused into the model through FiLM (Feature-wise Linear Modulation) layers or cross-attention, allowing the U-Net to adapt its behavior based on the prompt and noise level.

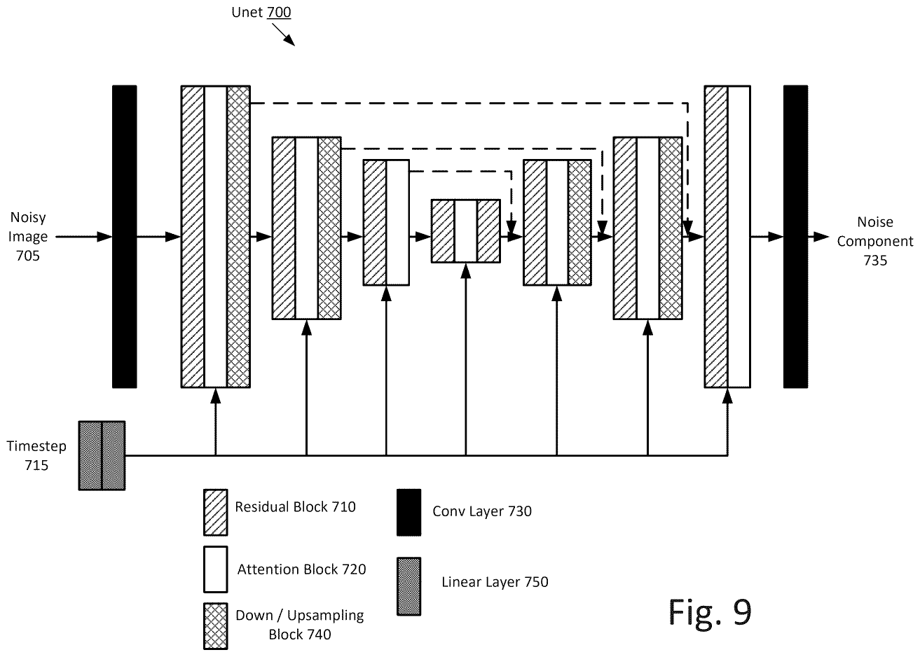

FIG. 9 depicts an example of an example architecture of a u-net that includes an encoder path and a decoder path. In the encoder path there are residual blocks (ResBlock) 710, attention blocks 720, and a down sampling operation 740 (max pooling). In the decoder path there are residual blocks 710, attention blocks 720, and up sampling 740. The timestep encodes the current timestep t (for example an integer from 1 to T, where T is the total number of diffusion steps) into a high-dimensional embedding. A linear layer 715 for the timestep is used to transform the timestep embedding. This embedding is then used to condition the U-Net, for example where each ResBlock 710 receives a time embedding. Each ResBlock 710 may be followed by an attention block 720. The attention mechanisms allow the network to apply external conditioning to the network.

In an embodiment, the model(s) are provided by or implemented with a neural network trained using deep learning. The network(s) may be defined as a plurality of sequential feature units or layers. Sequential is used to indicate the general flow of output feature values from one layer to input to a next layer. The information from the next layer is fed to a next layer, and so on until the final output. The layers may only feed forward or may be bi-directional, including some feedback to a previous layer. The nodes of each layer or unit may connect with all or only a sub-set of nodes of a previous and/or subsequent layer or unit. Skip connections may be used, such as a layer outputting to the sequentially next layer as well as other layers. Rather than pre-programming the features and trying to relate the features to attributes, the deep architecture is defined to learn the features at different levels of abstraction the input data. The features are learned to reconstruct lower level features (i.e., features at a more abstract or compressed level). For example, features for generating a fused image or higher resolution image are learned. For a next unit, features for reconstructing the features of the previous unit are learned, providing more abstraction. Each node of the unit represents a feature. Different units are provided for learning different features.

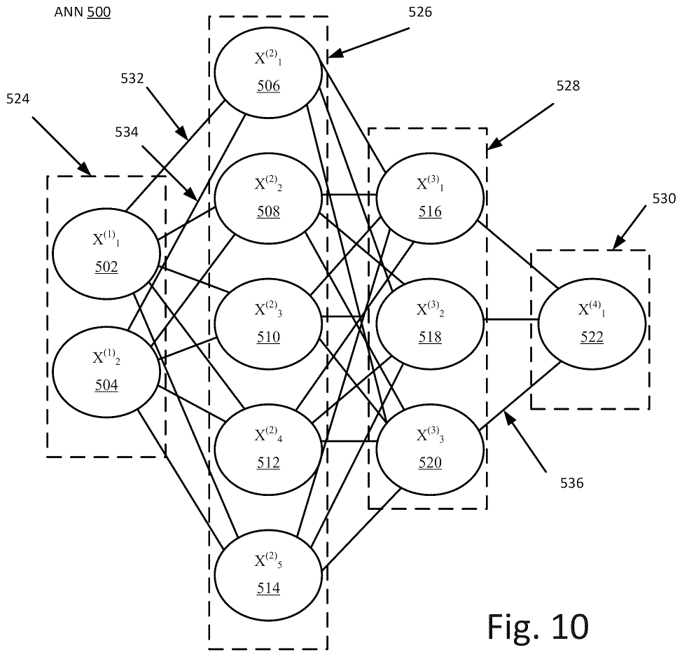

FIG. 10 shows an embodiment of an artificial neural network (ANN) 500, in accordance with one or more embodiments. Alternative terms for “artificial neural network” are “neural network”, “artificial neural net” or “neural net”. The artificial neural network 500 may be used in part in, for example, the one or more machine learning based networks utilized for the diffusion network(s), etc.

The artificial neural network 500 includes nodes 502-522 and edges 532, 534, . . . , 536, wherein each edge 532, 534, . . . , 536 is a directed connection from a first node 502-522 to a second node 502-522. In general, the first node 502-522 and the second node 502-522 are different nodes 502-522, it is also possible that the first node 502-522 and the second node 502-522 are identical. For example, in FIG. 10, the edge 532 is a directed connection from the node 502 to the node 506, and the edge 534 is a directed connection from the node 504 to the node 506. An edge 532, 534, . . . , 536 from a first node 502-522 to a second node 502-522 is also denoted as “ingoing edge” for the second node 502-522 and as “outgoing edge” for the first node 502-522.

In this embodiment, the nodes 502-522 of the artificial neural network 500 may be arranged in layers 524-530, wherein the layers may include an intrinsic order introduced by the edges 532, 534, . . . , 536 between the nodes 502-522. In particular, edges 532, 534, . . . , 536 may exist only between neighboring layers of nodes. In the embodiment shown in FIG. 10, there is an input layer 524 including only nodes 502 and 504 without an incoming edge, an output layer 530 including only node 522 without outgoing edges, and hidden layers 526, 528 in-between the input layer 524 and the output layer 530. In general, the number of hidden layers 526, 528 may be chosen arbitrarily. The number of nodes 502 and 504 within the input layer 524 usually relates to the number of input values of the neural network 500, and the number of nodes 522 within the output layer 530 usually relates to the number of output values of the neural network 500.

In particular, a (real) number may be assigned as a value to every node 502-522 of the neural network 500. Here, x⁽ⁿ⁾ᵢ denotes the value of the i-th node 502-522 of the n-th layer 524-530. The values of the nodes 502-522 of the input layer 524 are equivalent to the input values of the neural network 500, the value of the node 522 of the output layer 530 is equivalent to the output value of the neural network 500. Furthermore, each edge 532, 534, . . . , 536 may include a weight being a real number, in particular, the weight is a real number within the interval [−1, 1] or within the interval [0, 1]. Here, w^{(mn)}_{i,j} denotes the weight of the edge between the i-th node 502-522 of the m-th layer 524-530 and the j-th node 502-522 of the n-th layer 524-530. Furthermore, the abbreviation w⁽ⁿ⁾_{i,j} is defined for the weight w^{(n,n+1)}_{i,j}.

In particular, to calculate the output values of the neural network 500, the input values are propagated through the neural network. In particular, the values of the nodes 502-522 of the (n+1)-th layer 524-530 may be calculated based on the values of the nodes 502-522 of the n-th layer 524-530 by

$${x}_{j}^{( n + 1 )} = f ( {\sum }_{i} {x}_{i}^{( n )} \cdot {w}_{i , j}^{( n )} ) .$$

Herein, the function f is a transfer function (another term is “activation function”). Known transfer functions are step functions, sigmoid function (e.g. the logistic function, the generalized logistic function, the hyperbolic tangent, the Arctangent function, the error function, the smoothstep function) or rectifier functions. The transfer function is mainly used for normalization purposes.

In particular, the values are propagated layer-wise through the neural network, wherein values of the input layer 524 are given by the input of the neural network 500, wherein values of the first hidden layer 526 may be calculated based on the values of the input layer 524 of the neural network, wherein values of the second hidden layer 528 may be calculated based in the values of the first hidden layer 526, etc.

In order to set the values w^{(m,n)}_{i,j} for the edges, the neural network 500 has to be trained using training data. In particular, training data includes training input data and training output data (denoted as tᵢ). For a training step, the neural network 500 is applied to the training input data to generate calculated output data. In particular, the training data and the calculated output data include a number of values, said number being equal with the number of nodes of the output layer.

In particular, a comparison between the calculated output data and the training data is used to recursively adapt the weights within the neural network 500 (backpropagation algorithm). In particular, the weights are changed according to

$${w}_{i , j}^{′ ( n )} = {w}_{i , j}^{( n )} - γ \cdot {δ}_{j}^{( n )} \cdot {x}_{i}^{( n )}$$

wherein γ is a learning rate, and the numbers δ⁽ⁿ⁾ⱼ may be recursively calculated as

$${δ}_{j}^{( n )} = ( {\sum }_{k} {δ}_{k}^{( n + 1 )} \cdot {w}_{j , k}^{( n + 1 )} ) \cdot {f}^{′} ( {\sum }_{i} {x}_{i}^{( n )} \cdot {w}_{i , j}^{( n )} )$$

based on δ⁽ⁿ⁺¹⁾ⱼ, if the (n+1)-th layer is not the output layer, and

$${δ}_{j}^{( n )} = ( {x}_{k}^{( n + 1 )} - {t}_{j}^{( n + 1 )} ) \cdot {f}^{′} ( {\sum }_{i} {x}_{i}^{( n )} \cdot {w}_{i , j}^{( n )} )$$

if the (n+1)-th layer is the output layer 530, wherein f′ is the first derivative of the activation function, and y⁽ⁿ⁺¹⁾ⱼ is the comparison training value for the j-th node of the output layer 530.

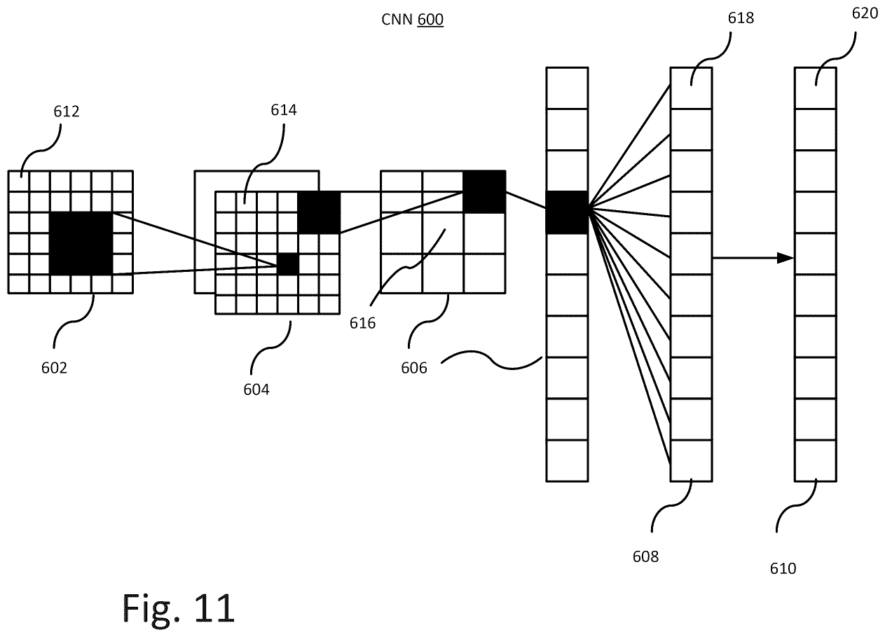

FIG. 11 shows a convolutional neural network (CNN) 600, in accordance with one or more embodiments. Machine learning networks described herein, such as, e.g., the diffusion network(s) etc. may be implemented using convolutional neural network 600.

In the embodiment shown in FIG. 11 the convolutional neural network 600 includes an input layer 602, a convolutional layer 604, a pooling layer 606, a fully connected layer 608, and an output layer 610. Alternatively, the convolutional neural network 600 may include several convolutional layers 604, several pooling layers 606, and several fully connected layers 608, as well as other types of layers. The order of the layers may be chosen arbitrarily, usually fully connected layers 608 are used as the last layers before the output layer 610.

In particular, within a convolutional neural network 600, the nodes 612-620 of one layer 602-610 may be considered to be arranged as a d-dimensional matrix or as a d-dimensional image. In particular, in the two-dimensional case the value of the node 612-620 indexed with i and j in the n-th layer 602-610 may be denoted as x⁽ⁿ⁾_{[i,j]}. However, the arrangement of the nodes 612-620 of one layer 602-610 does not have an effect on the calculations executed within the convolutional neural network 600 as such, since these are given solely by the structure and the weights of the edges.

In particular, a convolutional layer 604 is characterized by the structure and the weights of the incoming edges forming a convolution operation based on a certain number of kernels. In particular, the structure and the weights of the incoming edges are chosen such that the values x⁽ⁿ⁾ₖ of the nodes 614 of the convolutional layer 604 are calculated as a convolution x⁽ⁿ⁾ₖ=Kₖ*x⁽ⁿ⁻¹⁾ based on the values x⁽ⁿ⁻¹⁾ of the nodes 612 of the preceding layer 602, where the convolution * is defined in the two-dimensional case as:

$${x}_{k}^{( n )} [ i , j ] = ( {K}_{k} * {x}^{( n - 1 )} ) [ i , j ] = {\sum }_{{i}^{′}} {\sum }_{{j}^{′}} {K}_{k} [ {i}^{′} , {j}^{′} ] \cdot {x}^{( n - 1 )} [ i - {i}^{′} , j - {j}^{′} ] .$$

Here the k-th kernel Kₖ is a d-dimensional matrix (in this embodiment a two-dimensional matrix), which is usually small compared to the number of nodes 612-618 (e.g. a 3×3 matrix, or a 5×5 matrix). In particular, this implies that the weights of the incoming edges are not independent, but chosen such that they produce said convolution equation. In particular, for a kernel being a 3×3 matrix, there are only 9 independent weights (each entry of the kernel matrix corresponding to one independent weight), irrespectively of the number of nodes 612-620 in the respective layer 602-610. In particular, for a convolutional layer 604, the number of nodes 614 in the convolutional layer is equivalent to the number of nodes 612 in the preceding layer 602 multiplied with the number of kernels.

If the nodes 612 of the preceding layer 602 are arranged as a d-dimensional matrix, using a plurality of kernels may be interpreted as adding a further dimension (denoted as “depth” dimension), so that the nodes 614 of the convolutional layer 604 are arranged as a (d+1)-dimensional matrix. If the nodes 612 of the preceding layer 602 are already arranged as a (d+1)-dimensional matrix including a depth dimension, using a plurality of kernels may be interpreted as expanding along the depth dimension, so that the nodes 614 of the convolutional layer 604 are arranged also as a (d+1)-dimensional matrix, wherein the size of the (d+1)-dimensional matrix with respect to the depth dimension is by a factor of the number of kernels larger than in the preceding layer 602.

The advantage of using convolutional layers 604 is that spatially local correlation of the input data may exploited by enforcing a local connectivity pattern between nodes of adjacent layers, in particular by each node being connected to only a small region of the nodes of the preceding layer.

In the embodiment shown in FIG. 11, the input layer 602 includes 36 nodes 612, arranged as a two-dimensional 6×6 matrix. The convolutional layer 604 includes 72 nodes 614, arranged as two two-dimensional 6×6 matrices, each of the two matrices being the result of a convolution of the values of the input layer with a kernel. Equivalently, the nodes 614 of the convolutional layer 604 may be interpreted as arranged as a three-dimensional 6×6×2 matrix, wherein the last dimension is the depth dimension.

A pooling layer 606 may be characterized by the structure and the weights of the incoming edges and the activation function of its nodes 616 forming a pooling operation based on a non-linear pooling function f. For example, in the two dimensional case the values x⁽ⁿ⁾ of the nodes 616 of the pooling layer 606 may be calculated based on the values x⁽ⁿ⁻¹⁾ of the nodes 614 of the preceding layer 604 as

$${x}^{( n )} [ i , j ] = f ( {x}^{( n - 1 )} [ {id}_{1} , {jd}_{2} ] , ... , {x}^{( n - 1 )} [ i {d}_{1} + {d}_{1} - 1 , j {d}_{2} + {d}_{2} - 1 ] )$$

In other words, by using a pooling layer 606, the number of nodes 614, 616 may be reduced, by replacing a number d1·d2 of neighboring nodes 614 in the preceding layer 604 with a single node 616 being calculated as a function of the values of said number of neighboring nodes in the pooling layer. In particular, the pooling function f may be the max-function, the average, or the L2-Norm. In particular, for a pooling layer 606 the weights of the incoming edges are fixed and are not modified by training.

The advantage of using a pooling layer 606 is that the number of nodes 614, 616 and the number of parameters is reduced. This leads to the amount of computation in the network being reduced and to a control of overfitting.

In the embodiment shown in FIG. 11, the pooling layer 606 is a max-pooling, replacing four neighboring nodes with only one node, the value being the maximum of the values of the four neighboring nodes. The max-pooling is applied to each d-dimensional matrix of the previous layer; in this embodiment, the max-pooling is applied to each of the two two-dimensional matrices, reducing the number of nodes from 72 to 9.

A fully-connected layer 608 may be characterized by the fact that a majority, in particular, all edges between nodes 616 of the previous layer 606 and the nodes 618 of the fully-connected layer 608 are present, and wherein the weight of each of the edges may be adjusted individually.

In this embodiment, the nodes 616 of the preceding layer 606 of the fully-connected layer 608 are displayed both as two-dimensional matrices, and additionally as non-related nodes (indicated as a line of nodes, wherein the number of nodes was reduced for a better presentability). In this embodiment, the number of nodes 618 in the fully connected layer 608 is equal to the number of nodes 616 in the preceding layer 606. Alternatively, the number of nodes 616, 618 may differ.

A convolutional neural network 600 may also include a ReLU (rectified linear units) layer or activation layers with non-linear transfer functions. In particular, the number of nodes and the structure of the nodes contained in a ReLU layer is equivalent to the number of nodes and the structure of the nodes contained in the preceding layer. In particular, the value of each node in the ReLU layer is calculated by applying a rectifying function to the value of the corresponding node of the preceding layer.

The input and output of different convolutional neural network blocks may be wired using summation (residual/dense neural networks), element-wise multiplication (attention) or other differentiable operators. Therefore, the convolutional neural network architecture may be nested rather than being sequential if the whole pipeline is differentiable.

In particular, convolutional neural networks 600 may be trained based on the backpropagation algorithm. For preventing overfitting, methods of regularization may be used, e.g. dropout of nodes 612-620, stochastic pooling, use of artificial data, weight decay based on the L1 or the L2 norm, or max norm constraints. Different loss functions may be combined for training the same neural network to reflect the joint training objectives. A subset of the neural network parameters may be excluded from optimization to retain the weights pretrained on another datasets.

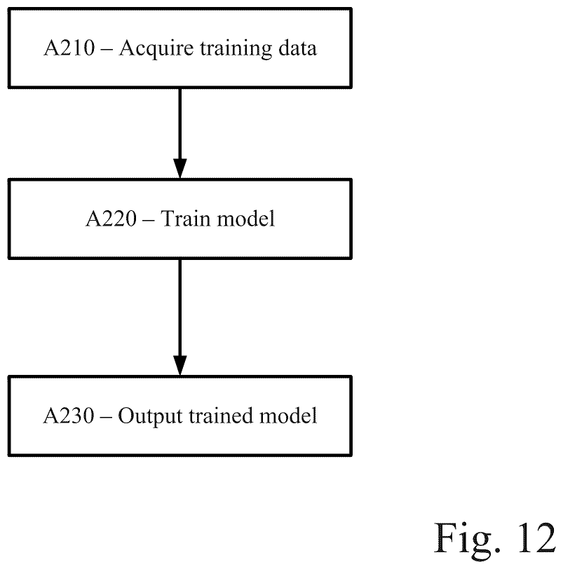

FIG. 12 depicts an example method for training the conditioned diffusion PnP Model 400. The method is performed by the system of FIG. 1 or another system. The method is performed in the order shown or other orders. Additional, different, or fewer acts may be provided. In an embodiment, the pretrained diffusion model is a previously trained neural network referred to as an PnP network. In an embodiment, the network is trained to learn the inverse diffusion process, for example to progressively recover clean images from noisy versions. The training of the network may be performed at any point prior to application. The training process starts with a dataset of high-quality images that are systematically corrupted by adding noise through a series of time steps. The goal of training is to teach the model to reverse this degradation and reconstruct the original images with high fidelity. In an embodiment, the training phase follows a modified diffusion framework where the model learns to predict the noise at each step, conditioned on the noisy input. The model is trained using a loss function, for example a mean squared error (MSE) between the predicted and true noise components, ensuring that the model accurately estimates the noise distribution. By minimizing this loss across multiple training examples, the model refines its ability to denoise images across different levels of corruption. The training involves optimizing an objective function, reminiscent of traditional denoising methods, which seeks to minimize the difference between the observed noisy image and the predicted clean image, adjusted by a prior that functions as a denoiser.

For training the conditioned diffusion PnP Model 400, at act A210, training data is acquired. The training data may include a dataset of medical image data that represents the style and subject matter that the diffusion model is configured to generate. Different sets of training data may be used for different adapters that are used for different purposes. For example, training data may include control maps for low b value images for DWI, noise maps, guidance/auxiliary images, derived maps from guidance/auxiliary images (e.g. edge maps, segmentation masks etc.), different acquisition protocol images (different contrasts, diffusion directions, structural scans such as T2w images in DWI), adjacent slices, etc.

At act A220, the adapted PnP Diffusion model is trained to estimate an image by finding the reverse transitions that maximize the likelihood of the training data. In an embodiment, the model is a generative model, in particular a diffusion model, for example, a DDPM or DDIM. In the learning phase, the forward process 210 learns the probability density function of MR image data by adding noise to the input image data. In the reverse process 210, an image is synthesized using the learned probability density function of MR image data. In an embodiment, the architecture of the conditioned diffusion PnP Model 400 includes the pretrained diffusion model and a trainable copy referred to as the adapter, for example as described in FIG. 6. The pretrained diffusion model is frozen (locked) during the training which keeps the original model's weights unchanged. The adapter 440 is initialized with the same weights, but with extra layers to process control conditions. Each block in the trainable U-Net is extended to accept a control signal, e.g. a control map. This signal is passed through zero convolution layers, which initially have no effect but learn to modulate the output over time.

During training, the model receives latent noised image (produced by adding Gaussian noise at a known timestep t), a control condition (e.g., images or maps relating to low b value images for DWI, noise maps, guidance/auxiliary images, derived maps from guidance/auxiliary images (e.g. edge maps, segmentation masks etc.), different acquisition protocol images (different contrasts, diffusion directions, structural scans such as T2w images in DWI), adjacent slices, etc.), and a ground truth image (used to compute the training loss). The latent noise image is passed through both the locked and trainable U-Nets. The conditional input is processed only by the trainable adapter 440 branch. In the forward pass, the latent noise goes through both branches of the U-Net. The trainable adapter 440 branch receives the control condition alongside the timestep embeddings. Outputs from the trainable and locked branches are fused (for example via addition or concatenation), allowing the control signal to influence the final prediction. The output is a prediction of the noise added to the latent (as in typical diffusion training). A loss function is used that takes into account the latent noise at timestep t, the actual added noise, the predicted noise, and the conditional input. The goal is to minimize the difference between the predicted and actual noise, just like standard diffusion training, but now under extra conditional guidance.

The control signal initially has no influence, thanks to zero conv layers of the adapter 440 branch. As the training progresses, these layers learn how to inject conditional structure into the model's output. Because the locked U-Net provides a strong prior, training is efficient even with smaller datasets.

At act A230, the trained model for denoising the MR image is output. The model may be applied to newly acquired MRI data in order to generate MR image data. In an embodiment multiple adapters may be trained, each for a different conditional input or parameter. In an example implementation, the adapted PnP diffusion model is used for DWI imaging. High-b value DWI suffer from low SNR. Existing deep learning methods for acquisition speed-up are sensitive to protocol changes. The diffusion prior is first trained on a diverse dataset. An adapter 440 is trained for high-b value DWI reconstruction using the low-b value DWI as condition. The usage of adapters improves upon the existing plug-and-play with diffusion methods, offering faster convergence. The proposed conditioned PnP diffusion model offers a flexible solution for using a pre-trained diffusion prior in a flexible framework for image reconstruction. Using the adapter 440 can improve certain scenarios, only if needed, without the need for re-training the prior.

In the implementation, for training the prior, a collection of more than 289K MR images of different body parts from scanners with different settings was used. The advantage of training a diverse prior instead of a specialized one is that the resulting foundation model can be used across inverse problems in multiple anatomical regions. For training the adapted PnP Diffusion model, brain DWI was used as a subset of the dataset. Paired low-b and high-b value DWI were used. Low-b DWI are used as control maps and the high-b DWI as input to the prior. Quantitative evaluation on retrospective accelerated scans and tasks like retrospective denoising or super-resolution is performed. By analyzing the results of the quantitative evaluation on the retrospective denoising and super-resolution tasks, it can be observed that using the low-b value DWI as control map for high-b DWI reconstruction outperforms in terms of PSNR and SSIM the Diffusion-PnP approach without the adapter. For accelerated scans scenario, the overall PSNR and SSIM are improved when using the adapter. Visual inspection of the results also reveals better quality. By inspecting intermediate steps of the adapted diffusion PnP algorithm, the adapter 440 is improving the prior's performance in the earlier iterations. This helps the later stages to leverage the generative power of the diffusion resulting in enhanced perceptual quality.

While the invention has been described above by reference to various embodiments, many changes and modifications can be made without departing from the scope of the invention. It is therefore intended that the foregoing detailed description be regarded as illustrative rather than limiting, and that it be understood that it is the following claims, including all equivalents, that are intended to define the spirit and scope of this invention. Independent of the grammatical term usage, individuals with male, female or other gender identities are included within the term.

The following is a list of non-limiting illustrative embodiments disclosed herein:

Illustrative embodiment 1: A method for image reconstruction of medical imaging data, the method comprising: acquiring a medical image of a patient; iteratively refining the medical image using a conditioned diffusion PnP model comprising a diffusion PnP model and an adapter, wherein the diffusion PnP model includes measurement during reverse diffusion steps that is carried out after a correction step that accounts for an inaccurate estimation resulting from computing a proximal solution, wherein the adapter comprises a neural network that adds conditions to the diffusion PnP model via finetuning; and outputting a refined medical image.

Illustrative embodiment 2. The method of illustrative embodiment 1, wherein the neural network of the adapter comprises an encoder that mirrors an encoder included in the diffusion PnP model.

Illustrative embodiment 3. The method of illustrative embodiment 1, wherein the adapter is configured to input a control map that comprises structural information relating to the acquisition of the medical image.

Illustrative embodiment 4. The method of illustrative embodiment 1, wherein the diffusion PnP model is pretrained, wherein for training the conditioned diffusion PnP model, weights of the diffusion PnP model are frozen while the adapter is trainable.

Illustrative embodiment 5. The method of illustrative embodiment 4, wherein for training the conditioned diffusion PnP model, zero initialization is used to halt an influence of harmful noise into hidden states of the adapter.

Illustrative embodiment 6. The method of illustrative embodiment 1, wherein the medical image is acquired using diffusion weight imaging.

Illustrative embodiment 7. The method of illustrative embodiment 6, wherein the adapter is configured to input higher SNR low b value images for conditioning high b value data that is reconstructed by the conditioned diffusion PnP model.

Illustrative embodiment 8. The method of illustrative embodiment 1, wherein the conditioned diffusion PnP model comprises multiple adapters, wherein the output of the multiple adapters is weighted and combined when used to condition the diffusion PnP model.

Illustrative embodiment 9. The method of illustrative embodiment 1, wherein the adapter is trained to condition the diffusion PnP model using at least one of noise maps, guidance/auxiliary images, derived maps from guidance/auxiliary images, edge maps, segmentation masks, different acquisition protocol images, textual input, or adjacent slices.

Illustrative embodiment 10. The method of illustrative embodiment 1, wherein the adapter is trained to condition the diffusion PnP model using textual information.

Illustrative embodiment 11. The method of illustrative embodiment 1, wherein the conditioned diffusion PnP model is configured to accommodate different user preferences for the reconstructed image through tuning Diffusion PnP hyperparameters that control a strength of a condition guidance and a level of noise injected at each timestep.

Illustrative embodiment 12. The method of illustrative embodiment 1, wherein the use of the adapter is halted when a user determines that no conditioning is required.

Illustrative embodiment 13. A system for image reconstruction of medical imaging data, the system comprising: a medical imaging system configured to acquire medical imaging data; a conditioned diffusion PnP model comprising a diffusion PnP model and an adapter, wherein the diffusion PnP model includes measurement during reverse diffusion steps that is carried out after a correction step that accounts for an inaccurate estimation resulting from computing a proximal solution, wherein the adapter comprises a neural network that adds conditions to the diffusion PnP model via finetuning; wherein the conditioned diffusion PnP model is further configured to input the medical imaging data and output a reconstructed medical image.

Illustrative embodiment 14. The system of illustrative embodiment 13, wherein the diffusion PnP model comprises a u-net architecture including an encoder and a decoder, wherein the neural network of the adapter comprises an encoder that mirrors the encoder included in the diffusion PnP model.

Illustrative embodiment 15. The system of illustrative embodiment 13, wherein the diffusion PnP model is pretrained, wherein for training the conditioned diffusion PnP model, weights of the diffusion PnP model are frozen while the adapter is trainable.

Illustrative embodiment 16. The system of illustrative embodiment 13, wherein the medical imaging system is configured to acquire the medical imaging data using diffusion weighted imaging.

Illustrative embodiment 17. The system of illustrative embodiment 16, wherein the adapter is configured to input higher SNR low b value images for conditioning high b value data that is reconstructed by the conditioned diffusion PnP model.

Illustrative embodiment 18. The system of illustrative embodiment 13, wherein the adapter is configured to condition the diffusion PnP model using at least one of noise maps, guidance/auxiliary images, derived maps from guidance/auxiliary images, edge maps, segmentation masks, different acquisition protocol images, textual input, or adjacent slices.

Illustrative embodiment 19. A method for training a conditioned diffusion PnP model, the method comprising: providing a pre-trained diffusion PnP model and an adapter network configured to input a control map; training the conditioned diffusion PnP model comprising the pre-trained diffusion PnP model and the adapter network, wherein weights of the pretrained diffusion PnP model are locked during the training which keeps the pretrained diffusion PnP model's weights unchanged while the adapter network's weights are changed; and outputting a trained conditioned diffusion PnP model.

Illustrative embodiment 20. The method of illustrative embodiment 19, wherein the adapter network is trained to condition the diffusion PnP model using at least one of noise maps, guidance/auxiliary images, derived maps from guidance/auxiliary images, edge maps, segmentation masks, different acquisition protocol images, textual input, or adjacent slices.

1. A method for image reconstruction of medical imaging data, the method comprising:
acquiring a medical image of a patient;
iteratively refining the medical image using a conditioned diffusion PnP model comprising a diffusion PnP model and an adapter, wherein the diffusion PnP model includes measurement during reverse diffusion steps that is carried out after a correction step that accounts for an inaccurate estimation resulting from computing a proximal solution, wherein the adapter comprises a neural network that adds conditions to the diffusion PnP model via finetuning; and
outputting a refined medical image.

2. The method of claim 1, wherein the neural network of the adapter comprises an encoder that mirrors an encoder included in the diffusion PnP model.

3. The method of claim 1, wherein the adapter is configured to input a control map that comprises structural information relating to the acquisition of the medical image.

4. The method of claim 1, wherein the diffusion PnP model is pretrained, wherein for training the conditioned diffusion PnP model, weights of the diffusion PnP model are frozen while the adapter is trainable.

5. The method of claim 4, wherein for training the conditioned diffusion PnP model, zero initialization is used to halt an influence of harmful noise into hidden states of the adapter.

6. The method of claim 1, wherein the medical image is acquired using diffusion weight imaging.

7. The method of claim 6, wherein the adapter is configured to input higher SNR low b value images for conditioning high b value data that is reconstructed by the conditioned diffusion PnP model.

8. The method of claim 1, wherein the conditioned diffusion PnP model comprises multiple adapters, wherein the output of the multiple adapters is weighted and combined when used to condition the diffusion PnP model.

9. The method of claim 1, wherein the adapter is trained to condition the diffusion PnP model using at least one of noise maps, guidance/auxiliary images, derived maps from guidance/auxiliary images, edge maps, segmentation masks, different acquisition protocol images, textual input, or adjacent slices.

10. The method of claim 1, wherein the adapter is trained to condition the diffusion PnP model using textual information.

11. The method of claim 1, wherein the conditioned diffusion PnP model is configured to accommodate different user preferences for the reconstructed image through tuning Diffusion PnP hyperparameters that control a strength of a condition guidance and a level of noise injected at each timestep.

12. The method of claim 1, wherein the use of the adapter is halted when a user determines that no conditioning is required.

13. A system for image reconstruction of medical imaging data, the system comprising:
a medical imaging system configured to acquire medical imaging data;
a conditioned diffusion PnP model comprising a diffusion PnP model and an adapter, wherein the diffusion PnP model includes measurement during reverse diffusion steps that is carried out after a correction step that accounts for an inaccurate estimation resulting from computing a proximal solution, wherein the adapter comprises a neural network that adds conditions to the diffusion PnP model via finetuning;
wherein the conditioned diffusion PnP model is further configured to input the medical imaging data and output a reconstructed medical image.

14. The system of claim 13, wherein the diffusion PnP model comprises a u-net architecture including an encoder and a decoder, wherein the neural network of the adapter comprises an encoder that mirrors the encoder included in the diffusion PnP model.

15. The system of claim 13, wherein the diffusion PnP model is pretrained, wherein for training the conditioned diffusion PnP model, weights of the diffusion PnP model are frozen while the adapter is trainable.

16. The system of claim 13, wherein the medical imaging system is configured to acquire the medical imaging data using diffusion weighted imaging.

17. The system of claim 16, wherein the adapter is configured to input higher SNR low b value images for conditioning high b value data that is reconstructed by the conditioned diffusion PnP model.

18. The system of claim 13, wherein the adapter is configured to condition the diffusion PnP model using at least one of noise maps, guidance/auxiliary images, derived maps from guidance/auxiliary images, edge maps, segmentation masks, different acquisition protocol images, textual input, or adjacent slices.

19. A method for training a conditioned diffusion PnP model, the method comprising:
providing a pre-trained diffusion PnP model and an adapter network configured to input a control map;
training the conditioned diffusion PnP model comprising the pre-trained diffusion PnP model and the adapter network, wherein weights of the pretrained diffusion PnP model are locked during the training which keeps the pretrained diffusion PnP model's weights unchanged while the adapter network's weights are changed; and
outputting a trained conditioned diffusion PnP model.

20. The method of claim 19, wherein the adapter network is trained to condition the diffusion PnP model using at least one of noise maps, guidance/auxiliary images, derived maps from guidance/auxiliary images, edge maps, segmentation masks, different acquisition protocol images, textual input, or adjacent slices.
